(19) United States
(12) Patent Application Publication
(10) Pub. No.: US 2009/0011001 A1
(43) Pub. Date: Jan. 8, 2009

(54) Manufacturing process for liposomal preparations

(76) Inventors: Zafeer Ahmad, Wadsworth, IL (US); Gopal Anyarambhatla, Decatur, IL (US); Sushil Prem, Wadsworth, IL (US); Imran Ahmad, Wadsworth, IL (US)

Correspondence Address: WOOD, PHILLIPS, KATZ, CLARK & MORTIMER, 500 W. MADISON STREET, SUITE 3800, CHICAGO, IL, 60661, US

(21) Appl. No.: 11/666,644

(22) PCT Filed: Oct. 28, 2005
(86) PCT No.: PCT/US05/38899

§ 371 Date: Sep. 16, 2008

Related U.S. Application Data
(60) Provisional application No. 60/623,451, filed on Oct. 29, 2004.

(51) Int. Cl. A61K 9/127; A61K 31/337

(52) U.S. Cl. 424/450; 514/449

(57) ABSTRACT

The present invention provides a manufacturing process for liposomal preparations comprising water-insoluble or hydrophobic active principals. In accordance with one aspect of the inventive method, at least one active principal and lipid fraction are dissolved in an organic solvent. This solution is then subjected to reduced pressure (vacuum) in a container with or with out inert packing to remove the organic solvent, thereby forming a puffy cake comprising the active principal or principals and lipid fraction. This puffy cake is then mixed with an aqueous solution, under controlled conditions suitable to form a bulk liposomal preparation. Because the active principal is imbedded in the lipid bilayer, removal of the aqueous solution is optional.

### CROSS-REFERENCE TO RELATED APPLICATIONS

This patent application claims the benefit of U.S. Provisional Patent Application No. 60/623,451, filed on Oct. 29, 2004, the disclosure of which is incorporated herein.

### FIELD OF THE INVENTION

The present invention relates to a method of making commercial quantities of liposome preparations with water-insoluble active principals. More particularly, the method comprises: (1) dissolving one or more film-forming lipids in an organic solvent with at least one active principal, (2) depositing the lipids by evaporation of the organic solvent, and (3) contacting the lipid deposit with an aqueous solvent.

### BACKGROUND OF THE INVENTION

Ethanol dilution, thin film hydration and reverse phase evaporation represent some of the conventional methods widely available for making liposomal formulations. Although effective on a small-scale basis, these methods lack the ability to produce commercial quantities of liposomal preparations with high entrapment efficiencies. For example, given the limitations of flask surface area, thin film hydration lacks the ability to produce batches of liposomal paclitaxel that exceed 50 liters.

In an attempt to address these limitations, U.S. Pat. No. 5,702,722 suggests a process for the commercial production of liposomal water-soluble drugs. Although successful for large-scale production, U.S. Pat. No. 5,702,722 fails to describe any such commercial process for water-insoluble or hydrophobic agents. Thus, a need exists for a method capable of producing commercial quantities of liposome preparations with water-insoluble or hydrophobic principals and capable of demonstrating high entrapment efficiencies.

### SUMMARY OF THE INVENTION

The present invention provides a manufacturing process for liposomal preparations comprising water-insoluble or hydrophobic active principals. In accordance with one aspect of the inventive method, at least one active principal and lipid fraction are dissolved in an organic solvent. This solution is then subjected to reduced pressure (vacuum) in a container with or without inert packing to remove the organic solvent, thereby forming a puffy cake comprising the active principal or principals and lipid fraction. This puffy cake is then mixed with an aqueous solution under controlled conditions suitable to form a bulk liposomal preparation. Because the active principal is imbedded in the lipid bilayer, removal of the aqueous solution is optional. The bulk liposomal preparation can be further processed by size fractionation or reduction, sterilization by membrane filtration, lyophilization or other treatment. Size reduction facilitates better disposition in the body and also enables sterile filtration through a 0.22 micron filter. In addition, lyophilization of the final product increases the shelf life of the liposomal preparation.

These and other advantages of the inventive method, as well as additional inventive features, will be apparent from the description of the invention provided herein.

### BRIEF DESCRIPTION OF THE DRAWINGS

FIG. 1 is a process flow diagram depicting the manufacturing process in accordance with the present invention;

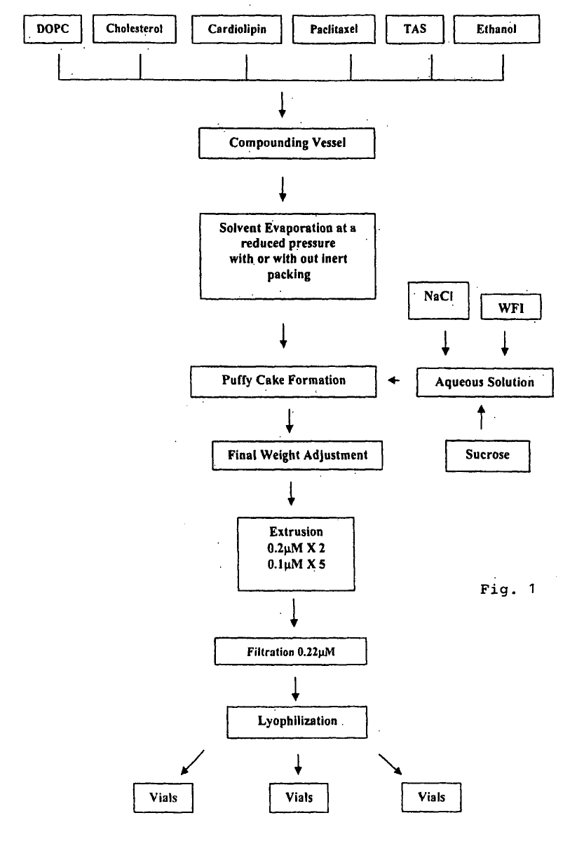

FIG. 2 is a histogram presenting the size distribution of paclitaxel containing liposomes after size reduction by extrusion and prior to lyophilization in accordance with the present invention; and

FIG. 3 is a histogram presenting the size distribution of paclitaxel containing liposomes after size reduction and lyophilization in accordance with the present invention wherein, prior to the size measurement, the lyophilized cake was reconstituted with the requisite amount of MilliQ water and measured for size.

### DETAILED DESCRIPTION OF THE INVENTION

The present invention provides a method of making a liposomal preparation with one or more water-insoluble entrapped active principals with an entrapment efficiency of about 80 to about 100 percent.

In accordance with the inventive method, an organic solvent is employed to dissolve a lipid fraction and one or more active principals. Preferably, ethanol is used as the organic solvent. The lipid fraction can comprise any suitable lipid or lipids capable of forming liposomes. Suitable lipids include pharmaceutically acceptable synthetic, semi-synthetic (modified natural) or naturally occurring compounds having a hydrophilic region and a hydrophobic region. Such compounds include amphiphilic molecules with net positive, negative or neutral charges or are devoid of any charge. Suitable lipids include compounds such as fatty acids and phospholipids, which can be synthetic or derived from natural sources, such as egg or soy. Suitable phospholipids include compounds such as phosphatidylcholine (PC), phosphatidylethanolamine (PE), phosphatidylserine (PS), phosphatidylglycerol (PG), phosphatidic acid (PA), phosphatidylinositol (PI), sphingomyelin (SPM) and the like, alone or in combination. Other suitable phospholipids include dimyristoylphosphatidylcholine (DMPC), dimyristoylphophatidylglycerol (DMPG), dioleoylphosphatidylglycerol (DOPG), distearoylphosphatidyl choline (DSPC), dioleoylphosphatidylcholine (DOPC), dipalmitoylphosphatidylcholine (DPPC), diarachidonoyl phosphatidylcholine (DAPC) or hydrogenated soy phosphatidylcholine (HSPC).

The lipid fraction can also include sterol and sterol derivatives such as cholesterol hemisuccinate (CHS), cholesterol sulfate and the like. Further, tocopherols and organic acid derivatives of tocopherols, such as α-tocopherol hemisuccinate, can also be used. Still further, the lipid fraction can also include polyethylene glycol derivatives of cholesterol (PEG-cholesterols), coprostanol, cholestanol, cholestane or α-tocopherol. Preferred lipids in the lipid fraction include one or more of cholesterol, dioleoylphosphatidylcholine (DOPC), tetramyristoyl cardiolipin, and tocopheryl acid succinate. In some embodiments, tetramyristoyl cardiolipin can be substituted with positively-charged cationic cardiolipins, such as 1,3-Bis-(1,2,-bistetradecyloxy-propyl-3-dimethylethoxyammoniumbromide)-propan-2-ol [(R)-PCL-2] and the like. Preferably, the lipid fraction includes at least two of these compounds and, more preferably, the lipid fraction includes all of these compounds.

According to some embodiments, an effective formulation can be prepared by the sequential addition of the lipids that form the lipid fraction into the organic solvent. More preferably, the method involves the sequential addition of DOPC, cholesterol, tetramyristoyl cardiolipin and tocopheryl acid succinate so as to dissolve each in the organic solvent.

The active principals include one or more hydrophobic or water-insoluble drugs. The water-insoluble or hydrophobic drugs include at least one antineoplastic or antifungal agent. Preferred active principals are taxanes or derivatives thereof, such as paclitaxel, docetaxel and related compounds (e.g. epothilones A and B, epothilone derivatives, etc.) and anticancer agents such as mitoxantrone, camptothecins and related molecules (such as, for example, 7-ethlyl-10-hydroxycamptothecin (i.e. SN-38), irinotecan, etc.) and derivatives thereof, doxorubicin, daunorubicin, methotrexate, tamoxien, toremifene, cisplatin, epirubicin, gemcitabine HCl, gemcitabine conjugates, bioactive lipids and other hydrophobic or water-insoluble chemotherapeutics useful for the treatment of cancer. Preferably, the active principal comprises at least one active principal selected from the group consisting of taxanes or derivatives. The most preferred active principal is paclitaxel.

Any amount of active principal can be employed. For example, where about 2 mg/ml of paclitaxel is used, the paclitaxel is dissolved in at least about 1.5 to about 20 percent of organic solvent relative to batch size (volume of the total liposomal preparation). In the preferred embodiment, the paclitaxel is dissolved in about 5 percent of organic solvent relative to batch size. In some embodiments, the amount of paclitaxel can exceed about 2 percent by volume, relative to batch size.

At least one or more water-insoluble or hydrophobic active principals are dissolved in the organic solvent. The active principals are preferably dissolved in the organic solvent at temperatures above about 40° C. or between about 40° C. and about 65° C. Further, according to the preferred procedure, the active principals are added to the organic solvent prior to the addition of the lipids. The temperature at which other active principals can be dissolved in organic solvents may vary depending on the properties of the respective active principals. It is within the ordinary skill of the art to select a suitable temperature for dissolution.

The solution, containing the active principal and lipid fraction dissolved in the organic solvent, is subjected to reduced pressure under controlled temperatures in order to evaporate the solvent. This can take place on a supported or unsupported structure. A supported structure comprises an inert porous material in the reaction vessel. The inert material includes any material with a large surface to volume ratio. The temperature and pressure conditions may vary depending on the properties of the organic solvent. It is within the ordinary skill of the art to select a suitable temperature and pressure for solvent evaporation. The resulting formation after solvent evaporation is a three-dimensional “puffy cake.”

For forming the bulk liposome preparation, an aqueous solution is added to the “puffy cake” with mixing (e.g. using a conventional mixer, such as those manufactured by Labmaster, for example), at between about 100 rpm to about 350 rpm while maintaining the temperature above about 35° C., such as between about 35° C. and about 45° C. The amount of aqueous solution can vary, but generally comprises the greatest percentage of volume for the batch size. Preferably, the amount of aqueous solution is at least 90 percent of the batch size and, more preferably, the amount of aqueous solution is at least about 93 percent to about 94 percent of the batch size.

The aqueous solution can also include one or more additional ingredients, such as sugars, tonicity adjusters and the like. Suitable tonicity adjusters include salts, preferably sodium chloride, and other agents known to those of the ordinary skill in the art. Tonicity adjusters can be present in any suitable amount. However, when present, the tonicity adjusters typically represent less than about 2% of the aqueous solution and, more typically, less than about 1% of the aqueous solution. Preferably, the aqueous solution contains a protective sugar (such as, trehalose, sucrose, maltose, lactose, glucose, dextran, mannitol and sorbitol as well as combinations thereof). One or more of the protective sugars can be present in any suitable amount. However, when present, the protective sugar adjusters typically represent at least about 5% of the solution, and generally less than about 20% of the aqueous solution (more typically less than about 15% of the aqueous solution). The most preferred aqueous solution is 20 percent sucrose solution.

The aqueous solution can also include one or more active principals. Such active principals are water-soluble and include antineoplastic agents and antifungal agents. Is it preferred that the bulk liposome preparation is size-reduced or extruded in order to render the liposomes more uniform. Cycles of extrusion are through suitably sized polycarbonate membrane filters using a suitably sized extruder. Preferably, the liposomes are size-reduced by extrusion through 0.2 μm and 0.1 μm polycarbonate filters at pressures typically up to about 800 psi. The mean size of the liposomal formulations can be, for example, about 120 nm to about 180 nm, preferably about 120 nm to about 150 nm and, more preferably, about 120 nm to about 130 nm, as measured by dynamic light scattering techniques.

It is preferred that the extruded liposomes are sterile-filtered. Preferably, the liposomes are passed through a sterile 0.22 μm filter to in order to remove all viable microbes from the liposome product. Sterile filtration is performed prior to filling the product in sterilized containers under aseptic conditions.

Further, following the preferred procedure, the extruded liposomes are lyophilized by using a suitable lyophilizer under controlled conditions. Preferably, the lyophilization comprises a series of thermal treatments with at least two drying cycles. More preferably, the extruded liposomes are loaded at ambient temperature and the temperature is ramped in at least two stages with the first thermal treatment held at a temperature and for a period of time sufficient to remove unbound water from the extruded liposomes and the second thermal treatment held at a temperature and for a period of time sufficient to remove bound water from the extruded liposomes. It is within the ordinary skill of art to optimize the temperature and step time duration.

### EXAMPLE

The example demonstrates the manufacturing process for liposomal preparations of the present invention. This example is provided as a further guide to the practitioner of ordinary skills in the art and is not to be construed as limiting the invention in any way.

### Preparation of Puffy Cake of Lipids and Drug

1, 2 Dioleoly-sn-glycero-3-phosphatidylcholine (DOPC), cholesterol and 1,1′,2,2′ tetramyristoyl cardiolipin (cardiolipin) along with paclitaxel and alpha-tocopheryl acid succinate (TAS) were dissolved in ethanol by heating the contents at 45° C. and with stirring. The resulting colorless thick syrup of lipids and drug was then transferred to either a lyophilizer or a vacuum chamber. The solvent was evaporated under controlled temperature and suitable pressure conditions. Mild boiling of contents was observed at the outset followed by frothing of the contents as the pressure was reduced. At the end of the solvent evaporation, a white colored puffy cake of lipids and drug was formed (LEP-ETU).

### Hydration of Puffy Cake and Extrusion of Bulk Liposomes

The puffy cake of lipids and drug was hydrated at room temperature with a suitable sugar solution containing sodium chloride for isotonicity under constant stirring. At the required pressure, the resulting liposomal formulation was then subjected to various cycles of extrusion using polycarbonate membrane filters of desired pore sizes (Whatman, Clifton, N.J.) and a suitably sized extruder (Lipex Biomembranes, Canada). The extruded liposome formulations were sterile-filtered and deposited into vials.

### Lyophilization of Filtered Liposomes

The extruded liposome formulations were lyophilized using a suitable lyophilizer under the following controlled conditions.

The thermal treatment was conducted over the course of six hours. First, the vials, containing the extruded liposomal formulations, were loaded at ambient temperature. Next, the shelf temperature was ramped to −5° C. over 60 minutes. (0.5°/min, 30°/hr). Then, the shelf temperature was ramped to −45° C. over 240 minutes. (0.17°/min, 10°/hr). The shelf temperature was then held at −45° C. for 60 minutes.

The liposomal formulations were then subjected to one round of drying over the course of 112 hours (6720 min). First, the shelf temperature was ramped from −45 to −25° C. over 60 minutes (0.33° C./min, 20°/hr) with vacuum at 100 microns. The shelf temperature was then held at −25° C. for 2880 minutes with vacuum at 100 microns. Next, the shelf temperature was ramped to −22° C. over 60 minutes (0.1° C./min, 60/hr) with vacuum at 100 microns. The shelf temperature was then held at −22° C. for 3720 minutes with vacuum at 100 microns.

After the first round of drying, the liposomal formulations were subjected to a second round of drying over the course of 18 hours (1080 min). First, the shelf temperature was ramped to 25° C. over 360 minutes (0.13° C./min, 80/hr) with vacuum at 100 microns. The shelf temperature was then held at 25° C. for 720 minutes with vacuum at 100 microns. Next, the shelf temperature was ramped to 5° C. over 40 minutes (0.5° C./min, 300/hr) with vacuum at 500 microns. The shelf temperature was then held at 5° C. with vacuum at 500 microns until stoppering. The total cycle time was 135 hours (5 days, 16 hours).

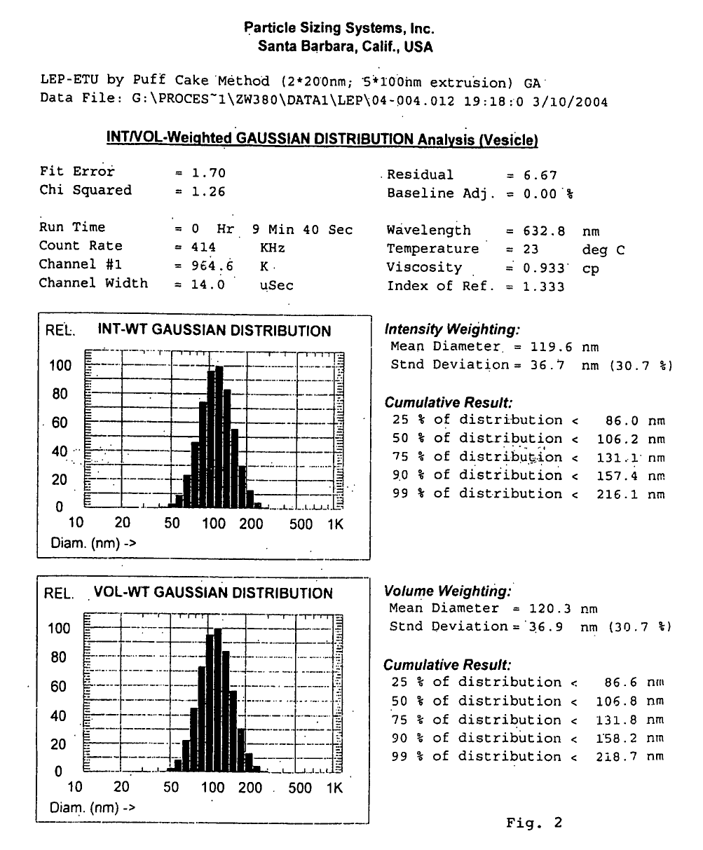

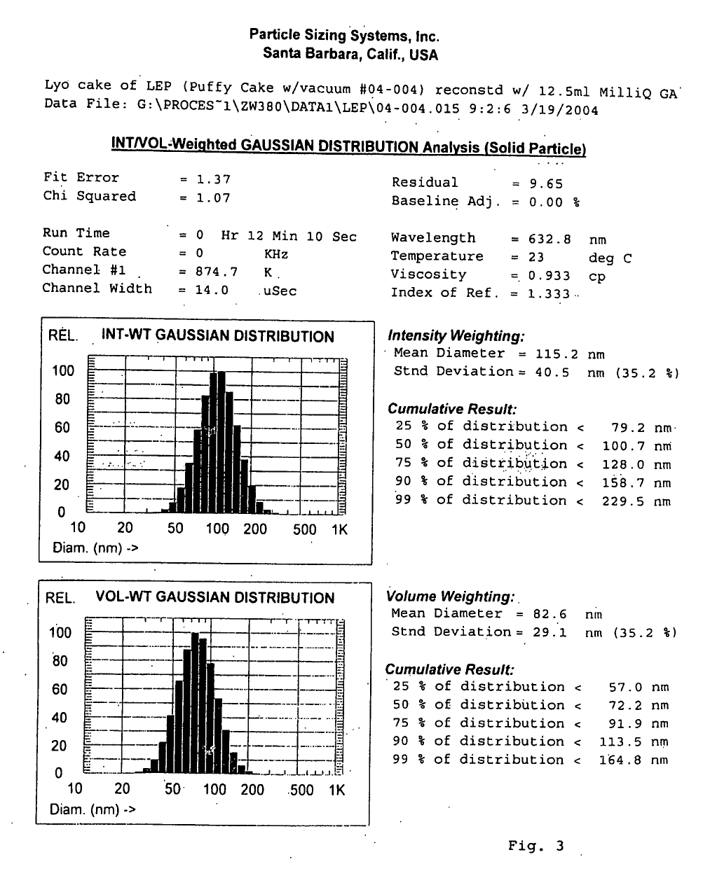

FIGS. 2 and 3 illustrate the particle size of pre-lyophilized and post-lyophilized liposomal samples. The pre-lyophilized suspension, after extrusion, showed a size of 120 nm (D-99 219 nm) with a chi squared value of 1.26, as shown in FIG. 2. The post-lyophilized cake reconstituted with requisite amount of MilliQ water showed a mean diameter of 115 nm (D-99 230 nm) with a chi squared value of 1.07, as shown in FIG. 3.

### Liposome Characterization

The extruded post-lyophilized liposomal formulations were characterized for parameters such as vesicle size, moisture content, lipid and drug content, entrapment efficiency, pH, among other parameters.

Mean vesicle diameter was measured by dynamic light scattering using the Nicomp Model 380 Sub-micron Particle Sizer (Particle Sizing Systems, Santa Barbara, Calif.). Polystyrene beads of standard size were used for instrument calibration and performance. The data was measured and reported on a volume-weighted distribution for vesicles.

The moisture content for the post lyophilized cake of LEP-ETU was determined using the Karl Fischer titrator (Mettler Toledo, Columbus, Ohio).

HPLC methods were used for the analysis of paclitaxel and lipid contents of LEP-ETU. Drug content analysis was performed using a Waters μ Bondapak C18, 39×300 mm, 10 μm HPLC column at 25° C. with a mobile phase of a mixture of acetonitrile and water (55/45, v/v) premixed at a flow rate of 1 mL/min. Sample injection volumes were 20 μL and paclitaxel detection was performed using a UV detector at a wavelength of 230 nm. DOPC and cholesterol were analyzed using an ASTEC DIOL HPLC column (Astec Inc., Whippany, N.J.) and an ELSD detector (Polymer Laboratories, Amherst, Mass.) at 40° C. with a chloroform:methanol:ammonium acetate buffer mobile phase at a flow rate of 1 mL/min. Sample injection volumes were 50 μL with evaporation and nebulization temperatures of 110° C. and 80° C., respectively. Cholesterol was analyzed using Hypersil BDS C18 (250 mm×4.6 mm, 5 μm) HPLC column with a mobile phase of acetonitrile:isopropanol (75:25, v/v) at 1.5 mL/min flow rate and 40° C. column temperature. Cholesterol detection was done using a UV detector at 205 nm.

Entrapment efficiency of paclitaxel in liposomes was determined by a mini-column centrifugation method using commercially available Sephadex G-25 columns (Macrospin Column, Harvard Biosciences, Holliston, Mass., USA). Briefly, Sephadex G-25 gel was allowed to swell in about 500 μL in MilliQ water for 15 minutes. The column was centrifuged for 4 minutes at 350×g using a table-top microfuge (Sorvall Biofuge fresco). The dry column was loaded with 100 μl placebo liposomes for LEP-ETU and centrifuged for 15 minutes at 1520×g to expel the liposomes. Subsequently, the LEP-ETU sample was introduced into the column and centrifuged at 1520×g for 15 minutes. The eluted sample was analyzed for entrapped paclitaxel concentration using HPLC compared with paclitaxel concentration in LEP-ETU prior to column chromatography to determine the entrapment efficiency.

These results were then compared to the results of LEP-ETU prepared by thin film hydration and an alternative puffy cake method. Table 1 shows a comparative profile of a cGMP sample of LEP-ETU (prepared by thin film hydration) along with two batches of LEP-ETU prepared using puffy cake method. The two batches made from puffy cake differ in the way the solvent was evaporated. For the batch # LEP-04-001, a lyophilizer was used to evaporate the solvent whereas for # LEP-04-004, a vacuum chamber was used to remove the solvent.

TABLE 1
Comparative profile for LEP-ETU (Thin film hydration v. Puffy
cake method)

| | | LEP-ETU | LEP-ETU by Puffy Cake | |
|---|---|---|---|---|
| | | (Thin Film) | Method (PCM) | |
| | Specific- | cGMP | PCM w/ Lyo | PCM w/ |
| Test | ation | #282I0903 | 04-001 | vacuum 04-004 |
| Paclitaxel (%) | >90% | 102 | 100 | n/a |
| DOPC (%) | 70-110% | 99 | 102 | n/a |
| Cholesterol (%) | 70-110% | 99 | 98 | n/a |
| Cardiolipin (%) | 70-110% | 87 | 103 | n/a |
| Appearance | White | Passed | Passed | Passed |
| | Cake | | | |
| Moisture (%) | Report | 0.77 | 2.33 | 1.89 |
| Reconstitution | Uniform | Passed | Passed | Passed |
| time | | | | |
| pH | Report | 4.34 | 4.37 | 4.34 |
| Mean Size | <400 nm | 134 | 103 | 115.3 |
| (nm) | | | | |
| Entrapment | >85% | 101 | 100 | n/a |
| (%) | | | | |

As illustrated in the above Table 1, parameters like moisture content, pH, entrapment efficiency, lipid and drug content of the LEP-ETU prepared using the puffy cake method in accordance with the present invention were comparable to the cGMP sample of LEP-ETU prepared using thin film hydration.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Of course, variations of those preferred embodiments will become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of manufacturing a liposomal preparation, said method comprising: a. dissolving a lipid fraction and at least one active principal in an organic solvent; b. removing the organic solvent to form a puffy cake; and c. contacting said puffy cake with an aqueous solution to form a bulk liposomal preparation.

2. The method of claim 1, wherein the lipid fraction comprises at least one lipophilic agent selected from a group consisting of phosphatidylcholine, phosphatidylethanolamine, phosphatidylserine, phosphatidylglycerol, phosphatidic acid, phosphatidylinositol, sphingomyelin, sterol, sterol derivatives, tocopherol, tocopherol derivatives, PEG-cholesterol, fatty acid, dimyristoylphosphatidylcholine, dimyristoylphophatidylglycerol, dioleoylphosphatidylglycerol, distearoylphosphatidyl choline, dioleoylphosphatidylcholine, dipalmitoylphosphatidylcholine, diarachidonoyl phosphatidylcholine, hydrogenated soy phosphatidylcholine, cardiolipin, cationic cardiolipin and mixtures thereof.

3. The method of claim 1, wherein the lipid fraction consists of DOPC, cholesterol, tetramyristoyl cardiolipin and tocopheryl acid succinate.

4. The method of claim 1, wherein the organic solvent is ethanol.

5. The method of claim 1, wherein the active principal is selected from a group consisting of antineoplastic agents and antifungal agents.

6. The method of claim 5, wherein the active principal is water-insoluble.

7. The method of claim 5, wherein the active principal is hydrophobic.

8. The method of claim 5, wherein the antineoplastic agent is selected from a group consisting of taxane, mitoxantrone, camptothecin, doxorubicin, daunorubicin, methotrexate, tamoxien, toremifene, cisplatin, epirubicin, gemcitabine HCl, gemcitabine conjugates, bioactive lipids and derivatives thereof.

9. The method of claim 8, wherein the taxane is paclitaxel.

10. The method of claim 1, wherein the active principal is dissolved in the organic solvent prior to the addition of the lipid fraction.

11. The method of claim 1, wherein the removal of the organic solvent comprises the reduction of pressure under controlled temperatures sufficient to evaporate the organic solvent.

12. (canceled)

13. The method of claim 1, wherein the aqueous solution comprises at least one protective sugar.

14. The method of claim 13, wherein the protective sugar is selected from a group consisting of trehalose, sucrose, maltose, lactose, glucose, dextran, mannitol, sorbitol and combinations thereof.

15. The method of claim 1, wherein the aqueous solution comprises at least one tonicity adjuster.

16. The method of claim 1, wherein the aqueous solution comprises at least one active principal.

17. (canceled)

18. The method of claim 1, wherein the contacting comprises mixing said puffy cake with said aqueous solution.

19. The method of claim 1, further comprising size reducing the bulk liposomal preparation to obtain a size-reduced liposomal preparation.

20. (canceled)

21. (canceled)

22. The method of claim 19, wherein the size reduction is achieved by extrusion of the bulk liposomal preparation at pressures up to about 800 psi.

23. The method of claim 1, further comprising sterile filtering of the liposomal preparation.

24. The method of claim 1, further comprising lyophilizing the liposomal preparation.
